(19) United States
(12) Patent Application Publication
(10) Pub. No.: US 2019/0113510 A1
(43) Pub. Date: Apr. 18, 2019

(54) BIOLOGICAL MATERIAL ANALYSIS DEVICE, BIOLOGICAL MATERIAL ANALYSIS SYSTEM, BIOLOGICAL MATERIAL SELECTION METHOD, BIOLOGICAL MATERIAL ANALYSIS PROGRAM, AND CELL CULTURE VESSEL

(71) Applicant: SONY CORPORATION, TOKYO (JP)

(72) Inventors: JUNJI KAJIHARA, TOKYO (JP); MASAHIRO MATSUMOTO, KANAGAWA (JP)

(21) Appl. No.: 16/099,869

(22) PCT Filed: Apr. 13, 2017
(86) PCT No.: PCT/JP2017/015062

§ 371 Date: Nov. 8, 2018

(30) Foreign Application Priority Data
May 18, 2016 (JP) 2016-099536

(51) Int. Cl. G01N 33/543

(52) U.S. Cl. CPC G01N 33/54373

(57) ABSTRACT

Provided are simplified and miniaturized cell analysis device and cell culture vessel for capturing a biological material such as a DNA or a cell two-dimensionally and further analyzing the biological material at high throughput with high sensitivity in a short time. A biological material analysis device or the cell culture vessel includes: a solid-state image sensor; and a molecule capable of bonding to a biological material, immobilized on a light-receiving surface of the solid-state image sensor via a stimulus-degradable linker.

### TECHNICAL FIELD

The present invention relates to a biological material analysis device, a biological material analysis system, a biological material selection method, a biological material analysis program, and a cell culture vessel.

### BACKGROUND ART

Recently, in regenerative medicine, it has been required to select a target cell with high purity and to culture the cell.

Examples of a method for selecting a cell include flow cytometry. In the flow cytometry, a cell is taken out from a culture substrate once and purified. However, there is a risk of damaging the cell, for example, and another method for selecting a cell is further demanded.

As such a method, for example, Patent Document 1 discloses a method for analyzing and discriminating a cell using a film of a cell adhesive light control material obtained by bonding a cell adhesive material to a cell non-adhesive material via a photodissociating group. According to this method, since the substrate can be irreversibly changed from a cell adhesive substrate to a non-adhesive substrate by a photodissociation reaction, adhesion selectivity between a cell and the substrate is excellent, and purity of the cell, a recovery ratio thereof, and the like can be increased.

In addition, not only a method for selecting a cell but also various methods for selecting another biological material, for example, a nucleic acid are being developed. Examples of the method for selecting a nucleic acid include a method using a DNA microarray, but a large device is used because a scanning mechanism is required. Therefore, in recent years, a method not requiring the scanning mechanism of the microarray has also been developed.

For example, Patent Document 2 discloses a DNA analysis chip including: a solid-state imaging device; an optical transmission unit for transmitting an image from one surface to the other surface, placed on a light-receiving surface of the solid-state imaging device such that the one surface faces the light-receiving surface of the solid-state imaging device; and a known DNA.

In addition, Non-Patent Document 1 discloses a droplet array for performing PCR amplification and fluorescence detection on a chip using an image in accordance with need for performing DNA analysis at high-throughput in a high-dynamic range.

### CITATION LIST

### Patent Document

Patent Document 1: PCT International Application Laid-Open No. 2011/058721
Patent Document 2: Japanese Patent Application Laid-Open No. 2006-71417

### Non-Patent Document

Non-Patent Document 1: “1-Million droplet array with wide-field fluorescence imaging for digital PCR”, Lab Chip, 2011 Nov. 21; 11(22):3838-45

### SUMMARY OF THE INVENTION

### Problems to be Solved by the Invention

However, further simplification of a device configuration, downsizing, automation, and the like are required for an analysis device. In addition, it is also desired to analyze a biological material such as a DNA or a cell at high throughput with high sensitivity in a shorter time. Furthermore, it is desired not to apply a stress such as damaging a biological material when a biological material is analyzed or selected.

In addition, in analysis using a DNA microarray, strong excitation light irradiation may be necessary in order to secure fluorescence intensity capable of detecting a labeled DNA. Therefore, an influence of phototoxicity on a detection target is concerned.

Furthermore, in a case where a DNA microarray is scanned and analyzed, time lag occurs due to scanning in observation time of the detection target on a DNA microarray surface.

### Solutions to Problems

In order to solve the above problem, the present technology provides a biological material analysis device including:

a solid-state image sensor; and

a molecule capable of bonding to a biological material, immobilized on a light-receiving surface of the solid-state image sensor via a stimulus-degradable linker.

The biological material analysis device of the present technology may have a spectral layer on the light-receiving surface of the solid-state image sensor.

In addition, the molecule capable of bonding to the biological material can be immobilized on the spectral layer.

The spectral layer can be formed on the light-receiving surface in a replaceable manner.

In addition, the spectral layer may be a color filter. As the color filter, a light absorption type color filter may be used.

As the solid-state image sensor, a CMOS may be used.

As the stimulus-degradable linker, a photodegradable linker may be used.

The molecule capable of bonding to the biological material may be selected from the group consisting of an oleyl group, an antibody, an aptamer, and a molecular recognition polymer.

In addition, the present technology provides a biological material analysis system, including:

a biological material capturing unit including: a solid-state image sensor; and

a molecule capable of bonding to a biological material, immobilized on a light-receiving surface of the solid-state image sensor via a stimulus-degradable linker; and

a light irradiation unit for irradiating the light-receiving surface of the solid-state image sensor with light.

The system may further include a biological material analysis unit for analyzing information regarding a biological material, obtained by the solid-state image sensor.

In addition, the present technology provides a biological material selection method for

applying a biological material-containing sample to a biological material capturing unit including:

a solid-state image sensor; and

a molecule capable of bonding to a biological material, immobilized on a light-receiving surface of the solid-state image sensor via a stimulus-degradable linker, and

selecting a desired biological material by analyzing information regarding a biological material captured by the molecule capable of bonding to a biological material, obtained by the solid-state image sensor.

The present technology further provides a biological material analysis program for causing a computer to implement an analysis function of analyzing a biological material captured by a molecule capable of bonding to a biological material, immobilized on a light-receiving surface of a solid-state image sensor via a stimulus-degradable linker.

In addition, the present technology further provides a cell culture vessel including:

a solid-state image sensor; and

a molecule capable of bonding to a biological material, immobilized on a light-receiving surface of the solid-state image sensor via a stimulus-degradable linker.

### Effects of the Invention

According to the present technology, a biological material can be captured two-dimensionally, and can be analyzed at once at high throughput with high sensitivity.

Note that the effects described here are not necessarily limited, and may be any of the effects described in the present disclosure.

### BRIEF DESCRIPTION OF DRAWINGS

FIG. 1 is a schematic diagram of a biological material analysis device according to the present technology.

FIG. 2 is a schematic diagram of a molecule capable of bonding to a biological material, immobilized on a solid-state image sensor according to the present technology.

FIG. 3 is a drawing substitute photograph of a bright field image of cells captured by the solid-state image sensor according to the present technology.

FIG. 4 is a drawing substitute photograph of a fluorescence image of cells captured by the solid-state image sensor according to the present technology.

FIG. 5 is a schematic diagram of a biological material analysis system according to the present technology.

FIG. 6 is a flowchart illustrating steps of cell culture using a cell culture vessel according to the present technology.

### MODE FOR CARRYING OUT THE INVENTION

Hereinafter, a preferred embodiment for carrying out the present technology will be described. Note that the embodiment described below exemplifies a representative embodiment of the present technology, and the scope of the present technology is not narrowly interpreted by the embodiment. The description will be made in the following order.

1. Biological material analysis device

2. Biological material analysis system

3. Biological material selection method

4. Biological material analysis program

5. Cell culture vessel

<1. Biological Material Analysis Device>

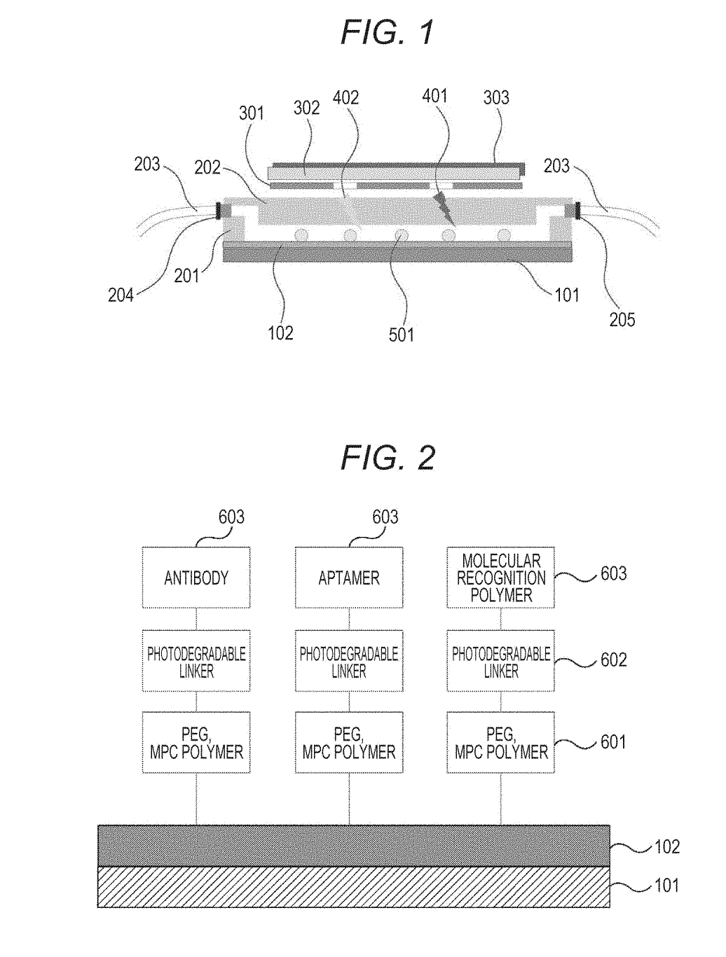

FIG. 1 illustrates an example of a biological material analysis device of the present technology.

A solid-state image sensor 101 captures a biological material captured on the solid-state image sensor as an image and analyzes the biological material.

As the solid-state image sensor 101, for example, a CMOS is preferably used. For example, if an effective area of the CMOS is 35.9 mm×24.0 mm and the number of effective pixels is about 42.4 million pixels, a pixel size is about 4.51 μm. A biological material is caused to exist on the CMOS, and the size of the biological material, the position where the biological material is captured, and the like are analyzed. For example, if the biological material is a cell, the size of, for example, about 10 μm in diameter can be captured with high sensitivity with a plurality of pixels.

As illustrated in FIG. 1, when a cell 501 is caused to exist on the solid-state image sensor 101, for example, it is possible to perform analysis based on how the cell 501 moves or flows, a position where the cell 501 is captured on the solid-state image sensor, a change in intensity of fluorescence emitted when the cell is labeled with fluorescence or the like, and a time lag in which phosphorescence or the like is emitted from the cell.

When the fluorescence is emitted by irradiation with excitation light, a spectral layer 102 may be disposed on a light-receiving surface of the solid-state image sensor 101. The spectral layer 102 can block excitation light as necessary and can transmit only weak fluorescence.

As the spectral layer 102, for example, a color filter can be used. With the color filter, a biological material can be analyzed using a plurality of discrimination elements. The entire surface of the solid-state image sensor can be used. Therefore, high throughput can be realized, and the sensitivity of analysis of a biological material can be enhanced. In order to perform analysis using a plurality of discrimination elements, for example, it is only required to use fluorescent labels of a plurality of colors for a captured biological material.

Examples of the color filter include an interference type color filter, a lamination type color filter, a light absorption type color filter, and a filter utilizing a surface plasmon resonance phenomenon by controlling the particle size of a gold nanoparticle. In the present technology, as described above, the light absorption type color filter is preferable for analyzing the size and the like of a cell using pixels although the present technology is not particularly limited thereto. The light absorption type color filter has such advantages that color resolution is good and a problem of crosstalk due to light emission from a plurality of cells can be solved, for example, as compared with the interference type and lamination type color filters.

In addition, the filter utilizing a surface plasmon resonance phenomenon by controlling the particle size of a gold nanoparticle changes the size of the gold nanoparticle, the shape thereof, chemical characteristics of a surface thereof, an aggregation state thereof, and the like, and can adjust absorption of light having a wavelength corresponding thereto.

The color filters and the filter utilizing a surface plasmon resonance phenomenon by controlling the particle size of a gold nanoparticle are not particularly limited. All pixels may be colored in a single color, or a plurality of fluorescent labels may be detectable by coloring respective pixels in different colors.

Note that emission of the fluorescence may be performed by a method such as chemiluminescence or electrochemiluminescence. In this case, the spectral layer 102 may be unnecessary or may be a transparent layer capable of transmitting light in the entire wavelength range.

Furthermore, a molecule capable of bonding to a biological material is immobilized on a light-receiving surface of the solid-state image sensor 101 via a stimulus-degradable linker.

FIG. 2 illustrates an example in which the spectral layer 102 is laminated on the light-receiving surface of the solid-state image sensor 101 and a molecule capable of bonding to a biological material is immobilized.

A surface of the spectral layer 102 or the light-receiving surface of the solid-state image sensor 101 may be coated with a substance (for example, collagen, fibroblast, or the like) in which a cell easily survives, but is not particularly limited.

In FIG. 2, a molecule 603 capable of bonding to a biological material is immobilized on the spectral layer 102 via a polymer 601 and a stimulus-degradable linker 602. The stimulus-degradable linker 602 may be directly immobilized without using the polymer 601.

In a case of using the polymer 601, preferably, for example, the polymer does not apply a stress to a cell, has no toxicity, and has biocompatibility. Examples of the polymer 601 include polyethylene glycol (PEG) and 2-methacryloyloxyethyl phosphorylcholine polymer (MPC polymer).

In a case of using the polymer 601, the stimulus-degradable linker 602 is bonded to the opposite end to a bond with the spectral layer 102 or the solid-state image sensor 101. A stimulus-degradable linker is a connecting molecule degraded by a specific external stimulus. Examples of the stimulus-degradable linker include a linker degraded by light of a specific wavelength, a linker degraded by an enzyme, a linker degraded by a temperature, and the like. The stimulus-degradable linker is not particularly limited. However, since a solid-state image sensor is used, a photodegradable linker can be selected.

The photodegradable linker is a molecule having a structure degraded by a specific wavelength.

Examples of the photodegradable linker include molecules having the following groups: a methoxy nitrobenzyl group, a nitrobenzyl group (Japanese Patent Application Laid-Open No. 2010-260831), a para-hydroxyphenacyl group (Tetrahedron Letters, 1962, volume 1, page 1), a 7-nitroindoline group (Journal of the American Chemical Society, 1976, volume 98, page 843), a 2-(2-nitrophenyl) ethyl group (Tetrahedron, 1997, volume 53, page 4247), a (coumarin-4-yl) methyl group (Journal of the American Chemical Society, 1984, volume 106, page 6860), and the like.

The wavelength at which the photodegradable linker is degraded almost coincides with the absorption wavelength of the molecule.

For example, in a case of a methoxy nitrobenzyl group used for the photodegradable linker, if absorption at 346 nm is assumed to be 1, absorption of 0.89, 0.15, and 0.007 are exhibited at 364 nm, 406 nm, and 487 nm, respectively. That is, degradation efficiency of the photodegradable linker is good with a light source of 365 nm, and the photodegradable linker is not substantially degraded with a light source of 488 nm.

In this manner, the wavelength of light emitted to the photodegradable linker only needs to be a wavelength corresponding to each photodegradable linker. Examples of the wavelength include a wavelength near a range of 330 to 450 nm. In addition, in a case where a biological material is a cell, light is preferably emitted, for example, at 30 mW/cm², 100 sec. →3 J/cm², which does not damage the cell. Particularly, it is preferable not to use a wavelength of 300 nm or less because the wavelength of 300 nm or less may damage a cell.

The molecule 603 capable of bonding to a biological material can be selected according to a biological material to be captured. For example, if the biological material to be captured is a DNA or an RNA, a complementary DNA or RNA can be selected as the molecule 603 capable of bonding to the biological material. If the biological material to be captured is a protein such as an antigen, an antigen specific antibody can be selected as the molecule 603 capable of bonding to the biological material. If the biological material to be captured is a cell, an oleyl group, an antibody, an aptamer, a molecular recognition polymer, or the like, capable of adhering to a cell surface, can be used as the molecule 603 capable of bonding to the biological material.

The oleyl group is hydrophobic, and for example, adheres to a cell surface. A spacer such as PEG may be added to the oleyl group, and a terminal thereof may include an N-hydroxysuccinimide group (NHS group).

The antibody bonds to a cell surface molecule antigen. Examples of the antibody include an antibody against a CD antigen appearing on a cell surface upon differentiation, an antibody against various cancer specific antigens, an antibody against major histocompatibility antigens, an antibody against a sugar chain, and the like.

The aptamer is a nucleic acid molecule or a peptide that specifically bonds to a molecule included in a cell to be captured. Examples of the aptamer include a DNA aptamer, an RNA aptamer, a peptide aptamer, a modified aptamer in which specificity is improved by introducing a modification into a nucleic acid skeleton or a base, and the like.

Even in the presence of a compound having physicochemical characteristics similar to a cell surface molecule of a cell to be captured, the molecular recognition polymer captures the target cell surface molecule with high selectivity. The molecular recognition polymer is also referred to as a molecular imprinted polymer, and has a selectively synthesized compound recognition region.

Note that a molecule capable of bonding to a biological material can be spotted in an array on the spectral layer 102.

As illustrated in FIG. 2 above, the molecule 603 capable of bonding to a biological material is immobilized on the spectral layer 102 laminated on the solid-state image sensor 101 via the polymer 601 and the photodegradable linker 602. Among these components, each of the spectral layer 102, the polymer 601, the photodegradable linker 602, and the molecule 603 capable of bonding to a biological material can be exchangeable, for example, as one sheet. If the single sheet is disposable and replaceable, the solid-state image sensor 101 can be repeatedly used.

When a sample containing a biological material is applied onto the single sheet, the biological material is captured by a specific bond. FIG. 1 schematically illustrates a case where the cell 501 is captured.

As illustrated in FIG. 1, for example, by disposing a vessel wall 201 and a vessel lid 202 on the solid-state image sensor 101, forming an inlet 204 and an outlet 205, and further disposing a tube 203, a sample containing the cell 501 can be applied. A sample flows from the inlet 204 to the outlet 205 to fill a vessel, and the cell 501 bonds to a molecule capable of bonding to a biological material immobilized on the spectral layer 102, for example, an antibody that bonds to a cell surface molecule (the molecule capable of bonding to a biological material is not illustrated in FIG. 1). A cell that has not bonded and an unnecessary substance can be removed by causing a buffer or the like to flow from the inlet 204.

Next, an antibody that bonds to another cell surface molecule is labeled with, for example, a fluorescent molecule as a second antibody, and can be applied from the inlet 204. The second antibody bonds to the cell 501 having an antigen against the second antibody to form a sandwich structure.

For example, in a case where a cell to be cultured is analyzed and selected, primary selection is possible by immobilizing an antibody against a specific CD antigen on a spectral layer and then applying a cell sample.

Next, secondary selection is possible by applying an antibody against another specific CD antigen as a fluorescently labeled second antibody to a cell after primary selection.

Furthermore, tertiary selection is possible by applying an antibody against another specific CD antigen as another fluorescently labeled third antibody to a cell after secondary selection.

In this way, by labeling antibodies against a plurality of kinds of CD antigens with different types of fluorescence, respectively, and using the antibodies, purity in detection and selection can be enhanced.

Alternatively, in a case where antibodies against a plurality of kinds of CD antigens are labeled with the same fluorescence, a plurality of the antibodies bonds to a cell, the intensity of the fluorescence can be enhanced, and detection can be performed with high sensitivity.

In any case, antibodies against a plurality of kinds of CD antigens may be applied separately or simultaneously.

Alternatively, by further combining use of an antibody that specifically bonds to the second antibody, an antibody that specifically bonds to the third antibody, and the like, the purity of a biological material to be selected can be enhanced.

In addition, simultaneously with the selection by the above method, the solid-state image sensor 101 can analyze a cell with an image. For example, the solid-state image sensor 101 analyzes the number of cells captured or the sizes of the cells, or selects the kind or the like of a cell with a plurality of types of fluorescence, and can perform analysis variously.

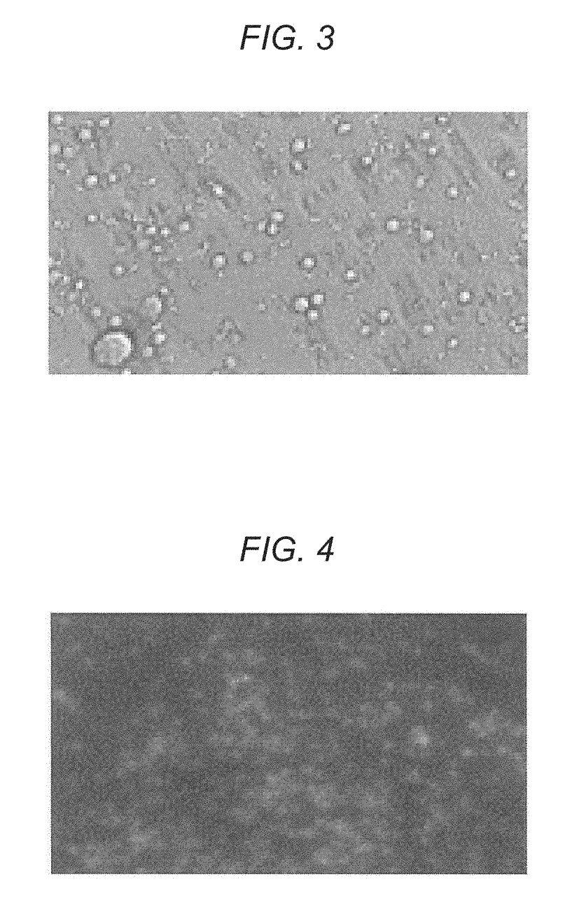

FIG. 3 illustrates an example of a bright field image of a captured cell by the solid-state image sensor, and FIG. 4 illustrates a fluorescence image. According to the present technology, it is possible to obtain various types of information regarding a cell from a bright field image and a fluorescence image without causing a time lag in a large area at once.

In addition, since a cell is captured on the solid-state image sensor, the detection sensitivity is higher than, for example, that of a conventional microscope. Alternatively, exposure time and irradiation with excitation light can be reduced as compared with a conventional device using a scanning mechanism, and an influence of phototoxicity on a cell can be reduced.

As illustrated in FIG. 1, irradiation with light can be selectively performed by disposing a light source 302 and a light source cover 303 and using a shutter 301 for an ultraviolet ray 401, visible light 402, and the like. Irradiation with light will be described later.

<2. Biological Material Analysis System>

The present technology provides a biological material analysis system including a biological material capturing unit including the above-described solid-state image sensor on which a molecule capable of bonding to a biological material is immobilized and a light irradiation unit including a light source of an ultraviolet ray, visible light, or the like.

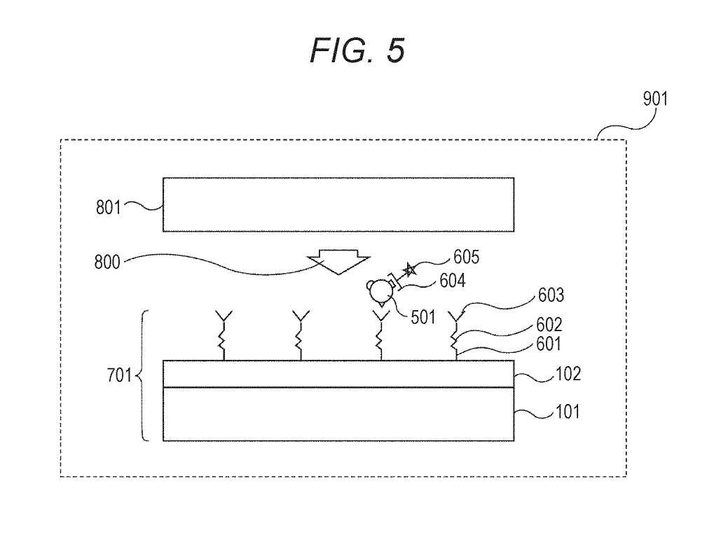

FIG. 5 illustrates an example of a biological material analysis system 901.

A biological material capturing unit 701 has a configuration in which the spectral layer 102 is laminated on the solid-state image sensor 101, and the polymer 601, the photodegradable linker 602, and the molecule 603 capable of bonding to a biological material, for example, an antibody that specifically bonds to a cell surface molecule, are immobilized on the spectral layer 102.

By applying a cell as a sample to the immobilized antibody, the cell 501 having a specific cell surface molecule is captured. An uncaptured cell is removed.

Next, by applying a second antibody 604 that specifically bonds to another specific cell surface molecule labeled with a fluorescent molecule 605 to the captured cell 501, the second antibody 604 specifically bonds to a cell having the other specific cell surface molecule among the captured cells 501.

A light irradiation unit 801 can emit light 800 having various wavelengths, and fluorescence is emitted, for example, by irradiating the fluorescent molecule 605 with specific excitation light. The fluorescence is spectrally dispersed by the spectral layer 102 which is, for example, a light absorption type color filter, and an image is obtained by the solid-state image sensor 101 which is, for example, a CMOS.

The biological material analysis system 901 can further include a biological material analysis unit for analyzing information regarding a captured biological material from an obtained image.

Examples of optical information obtained with an image include fluorescence intensity from a cell, a position where a cell has been captured, fluorescence duration time, and the like.

The image information is preferable for analyzing the number of captured biological materials, the sizes thereof, the kinds thereof, and the like, and can be analyzed at high throughput at a time. In addition, damage to a biological materials is less than that in conventional analysis using cell extraction by flow cytometry.

Specifically, a computer equipped with an image processing program can be used as the biological material analysis unit.

<3. Biological Material Selection Method>

The present technology can provide a method for selecting a biological material with high purity using the biological material analysis system.

First, a captured desired biological material is selected from analysis data by the biological material analysis unit. In a case where a substance other than a desired biological material is captured on a solid-state image sensor, stimulus is applied to a stimulus-degradable linker in this portion to release the captured substance. The released substance can be removed with a buffer or the like.

For example, in a case where the stimulus-degradable linker is a photodegradable linker, by irradiating the linker with light having a wavelength at which the linker is degraded from the light irradiation unit, the captured substance can be released and selected. In order to selectively irradiate a portion where a substance other than a desired biological material is captured with light, for example, a digital mirror device can be used.

<4. Biological Material Analysis Program>

The present technology also provides a biological material analysis program for causing a computer to implement an analysis function for analyzing the captured biological material.

The program may be stored in a recording medium such as a magnetic disk, an optical disk, a magneto-optical disk, or a flash memory, or may be distributed via a network, for example. Due to the program in such a form, analysis can be executed by externally attaching a computer to the biological material analysis device, or analysis can be executed by incorporating the computer in the biological material analysis device.

<5. Cell Culture Vessel>

As illustrated in FIG. 1, the biological material analysis device can be used also as a cell culture vessel by including the vessel wall 201 and the vessel lid 202.

That is, as described above, a substance other than a desired cell is removed, the vessel is filled with a medium while only the desired cell is held in the biological material analysis device, and cell culture can be performed as it is.

Since it is unnecessary to move a cell to be cultured to another vessel or the like for culturing, it is possible to reduce stress and damage to the cell.

In cell culture, in order to supply oxygen and to discharge carbon dioxide, the vessel lid 202 can be constituted by, for example, a material having gas permeability so as to obtain an environment suitable for cell culture (optimum CO₂ concentration, optimal temperature, and the like). In addition, the tube 203 may supply oxygen and discharge carbon dioxide.

Furthermore, a thermostatic device may be disposed such that the entire biological material analysis device illustrated in FIG. 1 has a thermostatic condition of 37° C.

For a medium, a medium suitable for a cell to be cultured can be selected, and examples of the medium include an Eagle's medium, a D-MEM medium, an E-MEM medium, an RPMI-1640 medium, a Dulbecco's PBS medium, and the like.

In addition, by coloring a medium with phenol red or the like, it is possible to control the medium in a pH optimum range (for example, pH 6.8 to 7.2) during culturing.

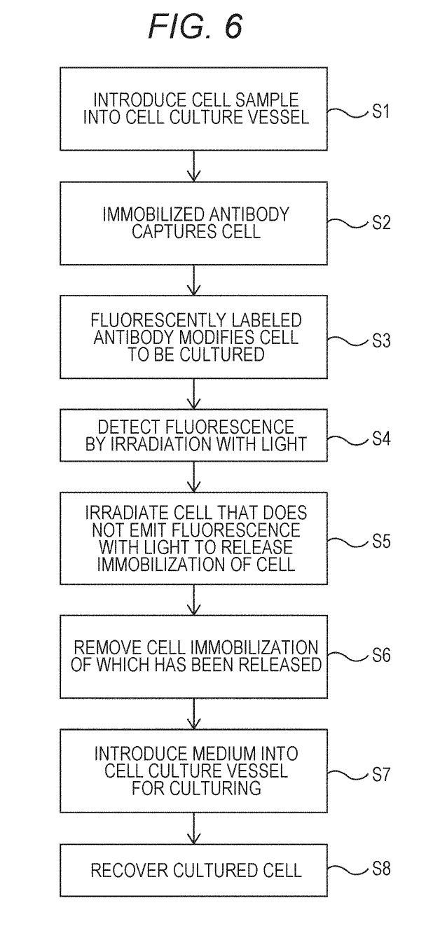

FIG. 6 illustrates an example of a step of cell culture in the present technology. Examples of a cell to be cultured include a cell differentiated from a stem cell.

First, a cell-containing sample is introduced into a cell culture vessel also serving as the biological material analysis device (S1). An antibody specific to a specific cell surface molecule is immobilized on the cell culture vessel via a photodegradable linker, and a cell is captured by the antibody (S2).

A cell that has not been captured and an unnecessary substance are removed with a washing solution such as a buffer.

Next, a fluorescently labeled antibody is introduced into the cell culture vessel and is further caused to bond to a cell to be cultured, and the cell to be cultured is modified with a fluorescent label (S3). A fluorescently labeled antibody that has not bonded is removed with a washing solution such as a buffer.

A fluorescent label modified with a cell is irradiated with light, and fluorescence is detected (S4). A cell that emits fluorescence is assumed to be a cell to be cultured. A cell that does not emit fluorescence is assumed to be a cell not to be cultured. In order to leave only a cell to be cultured in the cell culture vessel, a digital mirror device selectively irradiates a cell that does not emit fluorescence with light having a wavelength at which a photodegradable linker is degraded to degrade the linker, and immobilization is released (S5).

The released cell is removed with a washing solution such as a buffer (S6).

Incidentally, after removal, light having a wavelength at which the photodegradable linker is degraded may be further emitted to release an immobilized cell to be cultured, and then a subsequent culturing step may be performed.

A medium of a cell to be cultured is introduced into the cell culture vessel, and culturing is performed (S7). As culture conditions, a cell culture device may be placed under the culture conditions, or a device capable of adjusting the culture conditions, such as an oxygen supply device, a carbon dioxide discharge device, or a temperature control device, for example, may be included in the cell culture device.

During culturing, the number of cultured cells, the sizes thereof, the density thereof, the pH of a medium, and the like can be observed with an image. In addition, as for component analysis such as pH, detection may be performed with a micro electrode array or the like disposed outside a pixel region.

A cultured cell is recovered (S8). For example, if the cultured cell is a differentiated cell, the differentiated cell can be administered to a patient in need thereof. According to the present technology, a cultured cell with high purity can be produced. Therefore, a purification step is omitted, only a quality test is performed, and the cultured cell can be administered to a patient.

Incidentally, after a cultured cell is recovered, by removing the lid and the wall of the cell culture vessel, removing the spectral layer from the solid-state image sensor, newly attaching a spectral layer on which an antibody is immobilized via a photodegradable linker to the solid-state image sensor, and attaching a wall and a lid of the cell culture vessel again, a new cultured cell vessel can be obtained.

Note that the present technology can have the following configurations.
[1] A biological material analysis device including:

a solid-state image sensor; and

a molecule capable of bonding to a biological material, immobilized on a light-receiving surface of the solid-state image sensor via a stimulus-degradable linker.
[2] The biological material analysis device according to [1], having a spectral layer on the light-receiving surface of the solid-state image sensor.
[3] The biological material analysis device according to [2], in which the molecule capable of bonding to a biological material is immobilized on the spectral layer.
[4] The biological material analysis device according to [2] or [3], in which the spectral layer is formed on the light-receiving surface in a replaceable manner.
[5] The biological material analysis device according to any one of [2] to [4], in which the spectral layer is a color filter.
[6] The biological material analysis device according to [5], in which the color filter is a light absorption type color filter.
[7] The biological material analysis device according to any one of [1] to [6], in which the solid-state image sensor is a CMOS.
[8] The biological material analysis device according to any one of [1] to [7], in which the stimulus-degradable linker is a photodegradable linker.
[9] The biological material analysis device according to any one of [1] to [8], in which the molecule capable of bonding to a biological material is selected from the group consisting of an oleyl group, an antibody, an aptamer, and a molecular recognition polymer.
[10] A biological material analysis system, including:

a biological material capturing unit including:

a solid-state image sensor; and

a molecule capable of bonding to a biological material, immobilized on a light-receiving surface of the solid-state image sensor via a stimulus-degradable linker; and

a light irradiation unit for irradiating the light-receiving surface of the solid-state image sensor with light.
[11] The biological material analysis system according to [10], further including a biological material analysis unit for analyzing information regarding a biological material, obtained by the solid-state image sensor.
[12] A biological material selection method for

applying a biological material-containing sample to a biological material capturing unit including:

a solid-state image sensor; and

a molecule capable of bonding to a biological material, immobilized on a light-receiving surface of the solid-state image sensor via a stimulus-degradable linker, and

selecting a desired biological material by analyzing information regarding a biological material captured by the molecule capable of bonding to a biological material, obtained by the solid-state image sensor.
[13] A biological material analysis program for causing a computer to implement an analysis function of analyzing a biological material captured by a molecule capable of bonding to a biological material, immobilized on a light-receiving surface of a solid-state image sensor via a stimulus-degradable linker.
[14] A cell culture vessel including:

a solid-state image sensor; and

a molecule capable of bonding to a biological material, immobilized on a light-receiving surface of the solid-state image sensor via a stimulus-degradable linker.

### REFERENCE SIGNS LIST

101 Solid-state image sensor
102 Spectral layer
201 Vessel wall
202 Vessel lid
203 Tube
204 Inlet
205 Outlet
301 Shutter
302 Light source
303 Light source cover
401 Ultraviolet ray
402 Visible light
501 Cell
601 Polymer
602 Photodegradable linker
603 Molecule capable of bonding to biological material
604 Second antibody
605 Fluorescent molecule
701 Biological material capturing unit
800 Light
801 Light irradiation unit
901 Biological material analysis system

1. A biological material analysis device comprising:
a solid-state image sensor; and
a molecule capable of bonding to a biological material, immobilized on a light-receiving surface of the solid-state image sensor via a stimulus-degradable linker.

2. The biological material analysis device according to claim 1, having a spectral layer on the light-receiving surface of the solid-state image sensor.

3. The biological material analysis device according to claim 2, wherein the molecule capable of bonding to a biological material is immobilized on the spectral layer.

4. The biological material analysis device according to claim 2, wherein the spectral layer is formed on the light-receiving surface in a replaceable manner.

5. The biological material analysis device according to claim 2, wherein the spectral layer is a color filter.

6. The biological material analysis device according to claim 5, wherein the color filter is a light absorption type color filter.

7. The biological material analysis device according to claim 1, wherein the solid-state image sensor is a CMOS.

8. The biological material analysis device according to claim 1, wherein the stimulus-degradable linker is a photodegradable linker.

9. The biological material analysis device according to claim 1, wherein the molecule capable of bonding to a biological material is selected from the group consisting of an oleyl group, an antibody, an aptamer, and a molecular recognition polymer.

10. A biological material analysis system, comprising:
a biological material capturing unit including:
a solid-state image sensor; and
a molecule capable of bonding to a biological material, immobilized on a light-receiving surface of the solid-state image sensor via a stimulus-degradable linker; and
a light irradiation unit for irradiating the light-receiving surface of the solid-state image sensor with light.

11. The biological material analysis system according to claim 10, further comprising a biological material analysis unit for analyzing information regarding a biological material, obtained by the solid-state image sensor.

12. A biological material selection method for applying a biological material-containing sample to a biological material capturing unit including:
a solid-state image sensor; and
a molecule capable of bonding to a biological material, immobilized on a light-receiving surface of the solid-state image sensor via a stimulus-degradable linker, and
selecting a desired biological material by analyzing information regarding a biological material captured by the molecule capable of bonding to a biological material, obtained by the solid-state image sensor.

13. A biological material analysis program for causing a computer to implement an analysis function of analyzing a biological material captured by a molecule capable of bonding to a biological material, immobilized on a light-receiving surface of a solid-state image sensor via a stimulus-degradable linker.

14. A cell culture vessel comprising:
a solid-state image sensor; and
a molecule capable of bonding to a biological material, immobilized on a light-receiving surface of the solid-state image sensor via a stimulus-degradable linker.
